(19) United States
(12) Patent Application Publication
(10) Pub. No.: US 2021/0093375 A1
(43) Pub. Date: Apr. 1, 2021

(54) ABLATION LESION ASSESSMENT METHOD AND SYSTEM

(71) Applicant: SHANGHAI MICROPORT EP MEDTECH CO., LTD., Shanghai (CN)

(72) Inventors: Huimin CHU, Shanghai (CN); Huasheng CHENG, Shanghai (CN); Qingchun ZHANG, Shanghai (CN); Yahui PENG, Shanghai (CN); Liuping SHEN, Shanghai (CN); Yiyong SUN, Shanghai (CN)

(21) Appl. No.: 17/051,359

(22) PCT Filed: Apr. 24, 2019
(86) PCT No.: PCT/CN2019/084012

§ 371 Date: Oct. 28, 2020

(30) Foreign Application Priority Data
Apr. 28, 2018 (CN) 201810402737.4

(51) Int. Cl. A61B 18/14

(52) U.S. Cl. CPC A61B 18/1492; A61B 2018/00351

(57) ABSTRACT

An ablation lesion assessment method and system for obtaining ablation information by analyzing an intracardiac electrode signal from an ablation lesion are disclosed. The ablation lesion assessment system includes: a baseline calculation unit for generating a baseline for a signal profile and calculating a proportion of portions of the signal profile located above the baseline in the whole signal profile; a waveform comparison unit for obtaining a waveform comparison result by comparing the signal profile with an ablation pattern; and a determining unit for making a determination based on the proportion and the waveform comparison result and outputting the ablation information that indicates whether complete ablation has been achieved. The ablation lesion assessment method can utilize the ablation lesion assessment system to obtain the ablation information that indicates whether complete ablation has been achieved. This allows a surgeon to more easily know in real time during a cardiac radiofrequency ablation procedure, such as PVI, the progress in ablation or transmurality, thus making it possible to obtain a higher success rate and increased safety of the cardiac radiofrequency ablation procedure.

### TECHNICAL FIELD

The present invention relates to the field of medical equipments and, in particular, to an ablation lesion assessment method and system.

### BACKGROUND

Cardiac arrhythmia may occur as an abnormal rate and/or rhythm of the heartbeat due to the conduction of an abnormal sinoatrial (SA) node impulse or an impulse originating outside the SA node, which is delayed or blocked or takes place in an irregular pathway, i.e., an abnormality in the generation and/or propagation of cardiac electrical activity. For instance, atrial fibrillation (AF) is a tachycardia arrhythmia caused by the abnormal electrical signals.

Cardiac radiofrequency (RF) ablation has been widely used in the treatment of cardiac arrhythmias. This therapy utilizes various levels of energy to eliminate the source of an abnormal electrical signal by ablating the tissue that generates the signal, cutting off its pathway or destroying the abnormal tissue.

Cardiac radiofrequency ablation often leads to an ablated tissue region with transmural cell necrosis. Traditionally, for different lesion sites to be ablated, surgeons could determine, based on their experience, different values of the principal cardiac radiofrequency ablation parameters including RF power, ablation temperature and ablation time that control how the ablation carries on. However, this surgery remains risky for patients with complicated or unusual cardiac anatomy.

AF treatment is one important application of cardiac radiofrequency ablation. The pulmonary veins (PVs) have been widely recognized as AF trigger sites, and cardiac radiofrequency ablation for AF treatment, called pulmonary vein isolation (PVI), aims to ensure that there is not any single electrical connection between the pulmonary veins and the left atrium through electrically isolating triggers in the PVs.

PVI requires a continuous ablation lesion circumferentially around a pulmonary vein. However, since the PVs are complicated in anatomy and varying in wall thickness, ablation control is particularly critical for such procedures. Studies have found that electrical reconnection that may lead to AF recurrence or an iatrogenic atrial arrhythmia may happen, when effective isolation of the circumferential ablation lesion is not attained, e.g., with a tiny gap as small as 1 mm in the resulting scar-line around the pulmonary vein. On the contrary, the application of excessive ablation energy may bring damage to other organs such as the esophagus and phrenic nerve, or even cause cardiac perforation.

Therefore, there is a need for a method or apparatus that allows a higher success rate and improved safety of cardiac radiofrequency ablation procedures through real-time acquisition of ablation information without reliance on the surgeons' experience.

### SUMMARY OF THE INVENTION

The present invention aims to obtain, in real time, during a cardiac radiofrequency ablation procedure, information about whether complete ablation has been achieved, so as to allow the surgeon to know how the ablation proceeds in a timely manner and accordingly perform better control over the applied ablation energy, which can result in a higher success rate and increased safety of the procedure.

According to one aspect of the present invention, there is provided an ablation lesion assessment system for obtaining an ablation information by processing and analyzing an intracardiac electrode signal from an ablation lesion. The ablation lesion assessment system comprises: a signal processing module configured to process the intracardiac electrode signal and to generate a signal profile; and a signal analysis module comprising a determining unit, wherein the signal analysis module further comprises one or both of a baseline calculation unit and a waveform comparison unit. The baseline calculation unit is configured to generate a baseline for the signal profile, calculate a proportion of a portion of the signal profile located above the baseline in a whole signal profile and feed back the proportion to the determining unit. The waveform comparison unit is configured to obtain a waveform comparison result by comparing the signal profile with an ablation pattern associated with the ablation lesion, and feed back the waveform comparison result to the determining unit. The determining unit is configured to make a determination based on the proportion and/or the waveform comparison result and output the ablation information that indicates whether complete ablation has been achieved.

Optionally, the intracardiac electrode signal may vary over time, wherein the signal processing module is configured to obtain and process the intracardiac electrode signal in real time at a predetermined refresh rate and thereby generate a corresponding real-time signal profile. The refresh rate may be 1-2000 Hz.

Optionally, when the ablation information indicates that the complete ablation has been achieved, the determining unit may be configured to output the ablation information with a delay of a predetermined period of time after the determination is made. The predetermined period of time may be 1-10 seconds.

Optionally, when the proportion is equal to 100%, the ablation information may indicate that the complete ablation has been achieved, otherwise indicate that complete ablation has not been achieved. Alternatively, when the waveform comparison result indicates a similarity, the ablation information may indicate that complete ablation has been achieved, otherwise indicate that complete ablation has not been achieved.

Optionally, when the proportion is equal to 100%, the waveform comparison unit feeds back the waveform comparison result to the determining unit, and when the waveform comparison unit indicates a similarity, the ablation information indicates that the complete ablation has been achieved, otherwise indicates that the complete ablation has not been achieved.

Optionally, the waveform comparison unit may be configured to calculate a degree of waveform similarity between the signal profile and the ablation pattern associated with the ablation lesion, wherein when the degree of waveform similarity is greater than a predetermined threshold, the waveform comparison result indicates the similarity and otherwise indicates a dissimilarity.

Optionally, the ablation lesion may be associated with a plurality of ablation patterns, and the waveform comparison unit is configured to calculate Pearson a correlation coefficient between the signal profile and each of the plurality of ablation patterns and obtain an overall Pearson correlation coefficient by taking an average or a median of a plurality of Pearson correlation coefficients, wherein when the overall Pearson correlation coefficient is 0.6-1, the waveform comparison result indicates the similarity, otherwise indicates a dissimilarity, and wherein each of the plurality of Pearson correlation coefficients is calculated according to

$$P ( X , Y ) = \frac{Cov ( X , Y )}{{σ}_{X} {σ}_{Y}} ,$$

where P (X, Y) represents the Pearson correlation coefficient, X and Y denote X and Y coordinates of the signal profile and the ablation pattern, Cov(X,Y) is a covariance of X and Y, σ_{X} and σ_{Y} are standard deviations of X and Y.

Optionally, the ablation lesion may be associated with a plurality of ablation patterns, and the waveform comparison unit is configured to obtain the waveform comparison result by comparing the signal profile with the plurality of ablation patterns using a neural network algorithm.

Optionally, the ablation lesion assessment system may further comprise one or more of: an instruction reception module configured to activate or deactivate the ablation lesion assessment system and to set parameters of the ablation lesion assessment system; a signal input module configured to obtain and transmit the intracardiac electrode signal to the signal processing module; a display module and transmit display the ablation information; a voice module and transmit convey the ablation information; a storage module and transmit store the ablation pattern; and a pattern selection module and transmit determine an intracardiac site where the ablation lesion is being formed and to select the associated ablation pattern.

Optionally, the intracardiac electrode signal may be a single-electrode signal obtained by one electrode that is attached to an ablation catheter or an intracardiac mapping catheter and is brought into contact with the ablation lesion.

In another aspect of the present invention, there is provided an ablation lesion assessment method for obtaining an ablation information by analyzing an intracardiac electrode signal from an ablation lesion. The method comprises the steps of: generating a signal profile by processing the intracardiac electrode signal; generating a baseline for the signal profile, and calculating a proportion of a portion of the signal profile located above the baseline in a whole signal profile; and/or obtaining a waveform comparison result by comparing the signal profile with an ablation pattern associated with the ablation lesion; and making a determination based on the proportion and/or the waveform comparison result and outputting the ablation information that indicates whether a complete ablation has been achieved.

Optionally, making the determination based on the proportion and/or the waveform may comprise: when the proportion is equal to 100%, the ablation information indicates that the complete ablation has been achieved, otherwise indicates that the complete ablation has not been achieved; or when the waveform comparison result indicates a similarity, the ablation information indicates that the complete ablation has been achieved, otherwise indicates that the complete ablation has not been achieved.

Optionally, making the determination based on the proportion and/or the waveform comparison result may comprise: when the proportion is equal to 100%, the waveform comparison result is determined, and when the waveform comparison result indicates a similarity, the ablation information indicates that the complete ablation has been achieved, otherwise indicate that the complete ablation has not been achieved.

Optionally, when the ablation information indicates that the complete ablation has been achieved, outputting the ablation information a predetermined period of time after the determination is made.

The ablation lesion assessment system provided in the present invention is able to analyze, during a cardiac radiofrequency ablation procedure, an intracardiac electrode signal from an electrode brought into contact with an ablation lesion and thereby obtain ablation information indicative of whether complete ablation has been achieved. This ablation information can be provided to a surgeon so that he/she can know in real time the progress of ablation, e.g., whether the tissue has been transmural (i.e., complete ablation has been achieved). In this way, the cardiac radiofrequency ablation can be conducted in a more accurate manner. For example, in a PVI procedure, the ablation lesion assessment system can facilitate the formation of a circumferential ablation lesion without gaps or incompletely ablated portions. Moreover, even after the ablation process, the ablation lesion assessment system can be used to identify any gap in a timely manner and allows re-ablation. Thus, a significantly higher success rate and greatly increased safety of cardiac radiofrequency ablation can be obtained.

The ablation lesion assessment method provided in the present invention can be used to analyze an intracardiac electrode signal from an electrode brought into contact with an ablation lesion and thus obtain ablation information indicative of whether complete ablation has been achieved. With similar or the same advantages as the above-described ablation lesion assessment system, this method can be used to assess an ablation lesion with which the electrode is brought into contact before, during or after the ablation process.

The ablation lesion assessment system and method provided in the present invention can be implemented by a software program deployed outside the patient's body. The software program is stored in a computer readable medium and is applicable to any device or instrument that can display the intracardiac electrode signal, such as a multi-channel electrophysiological recorder, a three-dimensional mapping system or a radiofrequency ablation system. The electrode can be attached to a catheter, such as an ablation catheter or a mapping catheter, which has a distal end assuming a linear or annular shape or provided with a balloon. Ablation can be accomplished by RF energy, laser or freezing.

The ablation lesion assessment system and method provided in the present invention can also be implemented by a programmed computer system including a software program. Ablation information obtained, when the software program is executed, can help a surgeon more accurately control a cardiac radiofrequency ablation procedure so that a higher success rate and increased safety of the procedure can be achieved.

### BRIEF DESCRIPTION OF THE DRAWINGS

FIG. 1 schematically illustrates an electrode brought into contact with an ablation lesion in accordance with an embodiment of the present invention.

FIG. 2A is a graphical representation of an intracardiac electrode signal from an incompletely ablated lesion in accordance with an embodiment of the present invention.

FIG. 2B is a graphical representation of an intracardiac electrode signal from a completely ablated lesion in accordance with an embodiment of the present invention.

FIG. 3 is a schematic illustration of pulmonary vein isolation (PVI).

FIG. 4 is a diagram illustrating the structure of an ablation lesion assessment system in accordance with an embodiment of the present invention.

FIGS. 5(a) to 5 (d) are graphical representations of a signal profile in an ablation process in accordance with an embodiment of the present invention.

FIG. 6 schematically depicts a comparison drawn between a signal profile and an ablation pattern in accordance with an embodiment of the present invention.

FIG. 7 schematically illustrates ablation lesions for different intracardiac sites and corresponding ablation patterns in accordance with embodiments of the present invention.

FIG. 8 is a flowchart of an ablation lesion assessment method in accordance with an embodiment of the present invention.

FIG. 9 schematically illustrates the use of the method in an ablation process in accordance with an embodiment of the present invention.

In the figures,

20: an ablation lesion; 21: a transmural ablation lesion; 22: a nontransmural ablation lesion; 30: a pulmonary vein; 100: an ablation lesion assessment system; 110: a signal processing module; 120: a signal analysis module; 121: a baseline calculation unit; 122: a waveform comparison unit; 123: a determining unit; 130: an instruction reception module; 140: a signal input module; 150: a display module; 160: a storage module; and 170: a pattern selection module.

### DETAILED DESCRIPTION

Specific embodiments of the ablation lesion assessment system and method will be described in greater detail with reference to the accompanying drawings. Features and advantages of the invention will be more apparent from the following detailed description. Note that the figures are provided in a very simplified form not necessarily presented to scale, with their only intention to enhance convenience and clarity in explaining the disclosed embodiments. Furthermore, the terminology as used herein is for the purpose of describing the particular embodiments only and is not intended to be limiting of the invention. In this specification, singular references include the plural, unless the context clearly dictates otherwise. It will be further understood that the terms “comprises” and/or “comprising,” when used in this specification, specify the presence of stated compounds, ingredients, components, steps, operations, and/or elements, but do not preclude the presence or addition of one or more other compounds, ingredients, components, steps, operations, and/or elements thereof.

In addition, components in the present invention, such as logically separable software (computer programs), hardware or equivalents thereof, are generally referred to as “units” or “modules”. Therefore, “units” mentioned in the embodiments herein include both those in computer programs and those in hardware settings. Therefore, embodiments disclosed herein also include computer programs including instructions, which may be programs for carrying out each of steps in a computer, programs for implementing each computer function of as a tool, or programs for causing a computer to perform each of functions, systems or methods. “Units” or “modules” are activated by such computer programs. While the terms “storing”, “stored” or equivalents thereof can be used herein for ease of description, they are meant to describe the storage of programs or the storage of computer programs in storage devices in a controlled manner. Although individual “modules” and “units” can be configured in substantial one-to-one correspondence with respective functions, in practice, a single module may be configured to have one or more programs, or multiple modules may be configured to have either a single program or multiple programs. In a distributed or parallel environment, multiple modules may be implemented by a single computer, or a single module may be implemented by multiple computers. A single module may include additional modules.

Herein, an “apparatus” and/or “system” may include multiple computers, hardware elements, devices or the like that are interconnected by communication appliances in an injection network with one-to-one connectivity, or a single computer, hardware element, device or the like capable of implementing a process in accordance with the present invention.

Further, each process, whether performed by a single module or unit, or by multiple modules or units, may involve reading required information from storage devices such as a memory device and writing the results from the process into the storage devices. Therefore, for each process described herein, a description of reading from and writing into storage devices prior and subsequent to the process may be omitted. Herein, “storage devices” may include hard drive disks, random access memory (RAM) devices, external storage media, storage devices accessible via communication connections, registers in central processing units (CPUs), etc.

In a radiofrequency ablation procedure, an ablation electrode attached to a distal end of an ablation catheter can emit RF energy for ablating a tissue so that a signal cannot propagate from one side of the ablated tissue to the other. The present invention proposes an ablation lesion assessment system and method, in which an intracardiac electrode signal from an electrode brought into contact with an ablation lesion is processed and analyzed to obtain real-time information indicating whether complete ablation of the lesion has been achieved. The proposed system and method can be used in cardiac radiofrequency ablation and provide at least the following two benefits. First, the intracardiac electrode signal from the ablation lesion can be graphically displayed to provide the surgeon with an indication of whether the lesion has been completely ablated, i.e., transmural. Second, the surgeon can be indicated in real time of the lesion's condition before, during and after the ablation. Accordingly, more reasonable ablation parameters can be set and personalized ablation strategies with associated ablation parameters can be provided for different heart places. The system and method, as well as their benefits, will be described in detail below by way of specific embodiments.

An ablation lesion assessment system 100 according to an embodiment of the present invention allows obtaining an intracardiac electrode signal from an electrode brought into contact with an ablation lesion (referred to hereinafter as the “electrode E”, for the sake of clarity).

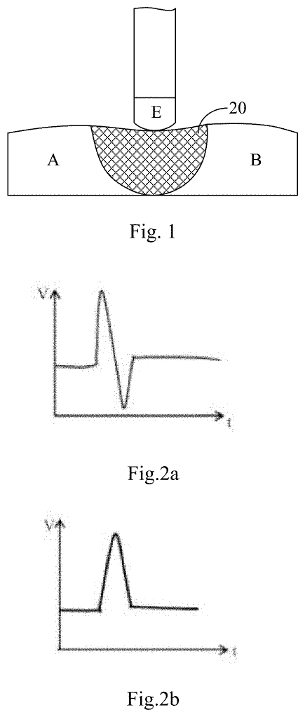

Specifically, the intracardiac electrode signal may be obtained by one or more electrodes brought into contact with the cardiac tissues. Preferably, the intracardiac electrode signal is obtained from one electrode that is disposed at a distal end of an ablation catheter or an intracardiac mapping catheter and brought into contact with a surface of the ablation lesion. That is, the intracardiac electrode signal is preferred to be a single-electrode signal. FIG. 1 schematically illustrates an electrode brought into contact with an ablation lesion in accordance with an embodiment of the present invention. FIG. 2(a) is a graphical representation of an intracardiac electrode signal from an incompletely ablated lesion in accordance with an embodiment of the present invention. FIG. 2(b) is a graphical representation of an intracardiac electrode signal from a completely ablated lesion in accordance with an embodiment of the present invention. Referring to FIG. 2(a), the intracardiac electrode signal obtained before complete ablation provides a voltage (V) signal profile over time (t) with a bipolar morphology with both positive portions corresponding to signal captured by the electrode E that is brought into contact with ablation lesion 20 from a side A of the electrode and negative portions corresponding to signal captured by the electrode E that is brought into contact with ablation lesion 20 from a side B of the electrode opposing the side A. With the ablation proceeding, the tissue at the ablation lesion 20 will become transmural and completely ablated, cutting off the path for interaction between electrical signals on both sides of the electrode E, as shown in FIG. 2(b), and the intracardiac electrode signal now exhibits a totally positive unipolar morphology.

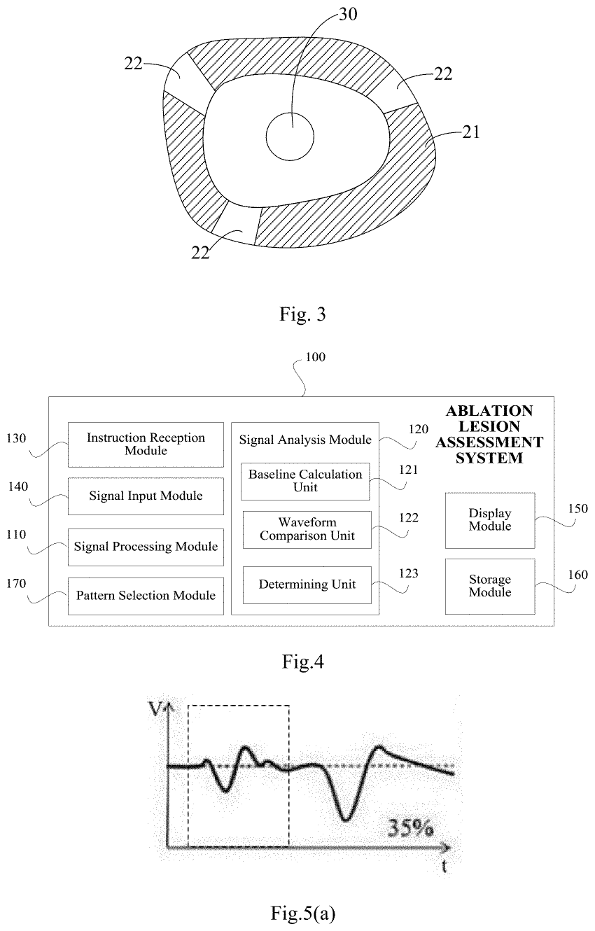

Using such a change in the intracardiac electrode signal fed back from the electrode E, assessment of ablation and transmurality is possible. With the use in pulmonary vein isolation (PVI) as an example, as schematically illustrated in FIG. 3, this procedure is required to form a continuous ablation circumferentially around each of one or more pulmonary veins 30. However, since the pulmonary vein 30 is complicated in anatomy and has different wall thicknesses at various sections, around the pulmonary veins 30, there may include both completely ablated transmural portions 21 and incompletely ablated nontransmural portions 22. The incompletely ablated nontransmural portions may lead to electrical reconnection and thus AF may recur or iatrogenic atrial arrhythmias may arise. Therefore, it is critical to control the ablation process so that the ablation lesion 20 is completely ablated and totally transmural without any gap therein. In accordance with embodiments of the present invention, an electrode E may be brought into contact with the ablation lesion 20 around the pulmonary vein 30 and an intracardiac electrode signal can be obtained therefrom. The signal profile of this intracardiac electrode signal can be constantly refreshed to enable real-time analysis and determination of whether a complete PVI has been obtained and whether the ablation is continuous.

An ablation lesion assessment system in accordance with an embodiment of the present invention will be described below in detail in the exemplary context of use in PVI.

This system is capable of processing the intracardiac electrode signal fed back from the electrode brought into contact with the ablation lesion and thereby obtaining ablation information. FIG. 4 shows the structure of the system. Referring to FIGS. 1 and 4, the ablation lesion assessment system 100 includes:

a signal processing module 110 configured to process the intracardiac electrode signal fed back from the electrode that is brought into contact with the ablation lesion and thereby generate a signal profile (referred to hereinafter as the “signal profile L”, for the sake of clarity); and

a signal analysis module 120 configured to analyze the signal profile L and thereby determine whether complete ablation has been achieved. To this end, the signal analysis module 120 may include a baseline calculation unit 121 and a determining unit 123 in some embodiments, or a waveform comparison unit 122 and the determining unit 123 in other embodiments, or the baseline calculation unit 121, the waveform comparison unit 122 and the determining unit 123 in still other embodiments. The signal analysis module is also configured to output ablation information indicating a result of the determination.

In some embodiments, the ablation lesion assessment system 100 may further include one or more of:

an instruction reception module 130 for activating/deactivating the ablation lesion assessment system 100 and configuring parameters thereof; a signal input module 140 for obtaining the intracardiac electrode signal from the electrode E and making it available to the signal processing module 110; a display module 150 for displaying the signal profile L and the ablation information; a storage module 160 for storing ablation pattern(s) associated with the ablation lesion 20, as well as the signal profile L, data generated during operation of the ablation lesion assessment system 100 and the ablation information indicating whether complete ablation has been achieved; and a pattern selection module 170 for determining the intracardiac site where the ablation lesion 20 is being formed and selecting the associated ablation pattern(s) based on the intracardiac site.

The intracardiac electrode signal fed back from the electrode E may be time-dependent, and the signal processing module 110 may refresh and process the signal at a predetermined rate so that the generated signal profile L is always based upon real-time recording. The signal analysis module 120 may analyze the real-time signal profile L by running either or both of the baseline calculation unit 121 and the waveform comparison unit 122, and output the ablation information indicating whether complete ablation has been achieved.

The signal input module 140 may be a multi-channel electrophysiological recorder, a three-dimensional mapping system, a radiofrequency ablation system or other equipment capable of capturing the intracardiac electrode signal, e.g., as an electrocardiogram (ECG or EKG), a graph of voltage (V, ordinate) versus time (t, abscissa).

Upon receiving the intracardiac electrode signal from the signal input module 140, the signal processing module 110 may filter it at 0.05-300 Hz and refresh it at the aforementioned predetermined rate. The predetermined refresh rate may be 1-2000 Hz, with 1 Hz corresponding to one-second electrocardiogram segments being preferred because it is close to the human heartbeat rate.

The signal processing module 110 may then automatically identify a segment of the intracardiac electrode signal (e.g., electrocardiogram) fed back from the electrode E as the signal profile L. Apart from the automatic identification by the ablation lesion assessment system, the segment as the signal profile L may also be selected by the surgeon. The signal profile L may have two time endpoints that are associated with a heartbeat frequency reflected by the intracardiac electrode signal. For instance, a unipolar signal segment over one heartbeat cycle of the electrocardiogram may be identified or selected as the signal profile L. Alternatively, a segment between points respectively of a predetermined period of time (e.g., 200 ms) preceding and succeeding a peak in a heartbeat cycle may be identified or selected as the signal profile L. However, the present invention is not limited to this, because the segment as the signal profile L may be determined otherwise according to other methods known in the art.

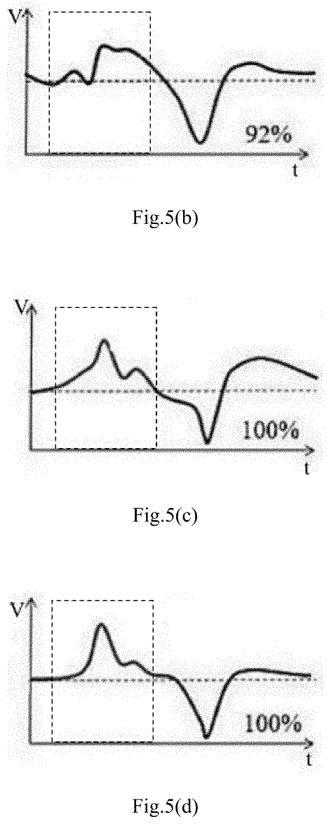

FIGS. 5 (a) through 5 (d) are graphical representations of a signal profiles in an ablation process according to an embodiment of the present invention. Specifically, the four graphical representations of FIGS. 5(a), 5(b), 5(c) and 5(d) are interceptions starting at respective time instants of an intracardiac electrode signal that evolves over time during the ablation process. In each of these figures, the signal profile L is encircled by a dashed box, and a dashed straight line marking a baseline for the signal profile L is shown. Operations of the baseline calculation unit 121 according to embodiments of the present invention will be explained in detail with reference to FIGS. 5(a) to 5(d).

Analysis of the baseline calculation unit 121 on the signal profile L may involve: generating the baseline for the signal profile L by the baseline calculation unit 121; and calculating a proportion of positive portions located above the baseline in the whole signal profile L and feeding back the proportion to the determining unit 123.

Further, generating the baseline may include the steps of: (1) normalizing the signal profile L: firstly, collecting multiple adjacent sampling points (i.e. voltage value captured at certain time), the number of the sampling points depends on the sampling frequency, e.g., 512 per second at 512 Hz, then defining a set of sampling points (e.g., 10) as a process window, hence to calculate the average value or weighted average value of each process window, thereby obtaining corresponding voltage value; (2) obtaining slopes by subtracting one of the voltage values of every adjacent two of the process windows from the other; and (3) setting the voltage value of the process window with the greatest slope as the baseline.

The generation of the baseline may also be accomplished with any of other common methods, such as finding the derivative of a fitted curve, wavelet transform, etc., a further detailed description of which is, however, omitted herein.

After the baseline has been generated for the signal profile L, a proportion of positive portions of the signal profile L located above the baseline in the whole signal profile L may be derived. This proportion can be calculated, e.g., as a ratio of the total area enclosed by the positive portions and the abscissa to that enclosed by the whole signal profile L and the abscissa. However, the present invention is not limited to this, as any form of the proportion of positive portions in the whole signal profile L is possible as long as it is obtained following the same convention.

In embodiments, the proportions of positive portions in the signal profiles L encircled in the respective dashed boxes in FIGS. 5(a), 5(b) and 5(c) may be calculated as 35%, 92% and 100%, respectively. FIG. 5(d) shows a signal profile L delayed 5 seconds from the signal profile L of FIG. 5(c). The proportion may be output to the determining unit 123.

Next, operations of the waveform comparison unit 122 according to embodiments of the present invention will be explained in detail.

The waveform comparison unit 122 may analyze the signal profile L, and the analysis may include: obtaining a waveform comparison result through comparing the signal profile L with the ablation pattern(s) associated with the ablation lesion 20 by the waveform comparison unit 122; and feeding back the waveform comparison result to the determining unit 123.

The ablation lesion assessment system 100 may be prescribed or stored in the storage module 160 with one or more ablation patterns (referred to hereinafter as the “ablation pattern(s) M”, for the sake of clarity). The ablation pattern(s) M may be, for example, signal profile(s) in successful ablation case(s). A single type of ablation lesion 20 (see FIG. 1) may be associated with one or more prescribed or stored ablation pattern(s) M. A comparison of the signal profile L with the ablation pattern(s) M by the waveform comparison unit 122 may result in a single waveform comparison result indicative of, for example, similarity or dissimilarity therebetween. If the waveform comparison result indicates similarity, then it is determined that the lesion has become transmural, i.e., completely ablated. The waveform comparison result may be output to the determining unit 123. Here, “similarity” means that the signal profile L has a pattern that is nearly the same as or identical to the ablation pattern(s) M.

For an ablation lesion 20 being created at an intracardiac site that is associated with a plurality of ablation patterns M, the waveform comparison unit 122 may calculate a Pearson correlation coefficient P (X, Y) between the signal profile L and each of the ablation patterns M according to

$$P ( X , Y ) = \frac{Cov ( X , Y )}{{σ}_{X} {σ}_{Y}} ,$$

where X and Y denote X- and Y-coordinates associated with the signal profile and the ablation pattern M, Cov(X, Y) is the covariance of X and Y, and σ_{X} and σ_{Y} are the standard deviations of X and Y. The waveform comparison unit 122 may additionally obtain an overall Pearson correlation coefficient by taking the average or median of the resulting Pearson correlation coefficients. Specifically, if the overall Pearson correlation coefficient is less than 0.4, it may be determined that the signal profile L is weakly correlated or uncorrelated with the ablation patterns M. If the overall Pearson correlation coefficient is within the range of 0.4-0.6 (here, mathematically denoted as the interval [0.4, 0.6) or [0.4, 0.6]), it may be determined that the signal profile L is moderately correlated with the ablation patterns M. If the overall Pearson correlation coefficient is within the range of 0.6-1 (here, mathematically denoted as the interval [0.6, 1] or (0.6, 1], which continues with the foregoing interval without any overlap therewith), it may be determined that the signal profile L is strongly correlated with the ablation patterns M, satisfying the similarity criterion. In some embodiments, when the overall Pearson correlation coefficient is within the range of 0.6-1, the waveform comparison result indicates similarity. Otherwise, the waveform comparison result may indicate dissimilarity. Those of ordinary skill in the art will know how to calculate the Pearson correlation coefficients from publications, and a further detailed description thereof is omitted herein.

For an ablation lesion 20 being formed at an intracardiac site that is associated with a plurality of ablation patterns M, the waveform comparison unit 122 may also obtain a waveform comparison result by utilizing a neural network algorithm to compare the signal profile L with the ablation patterns M. In this case, the number of layers in the neural network is preferred to be equal to or greater than 10, and the ablation patterns M used in the neural network may be signal profiles in historical successful ablation cases, which have been calibrated by experts. The neural network may be trained with a set with a size preferably of greater than or equal to 5,000 signal profile patterns and then used to generate the output waveform comparison result. During the ablation process, the waveform comparison unit 122 may output one waveform comparison result to the determining unit 123 at every unit time interval (e.g., one second). The neural network algorithm may be any of existing algorithms known in the fields of pattern comparison and image processing, and a further detailed description thereof is omitted herein.

The waveform comparison unit 122 may also obtain the aforementioned waveform comparison result by using any of other suitable algorithms such as wavelet transform, speeded up robust features and Frechet distance.

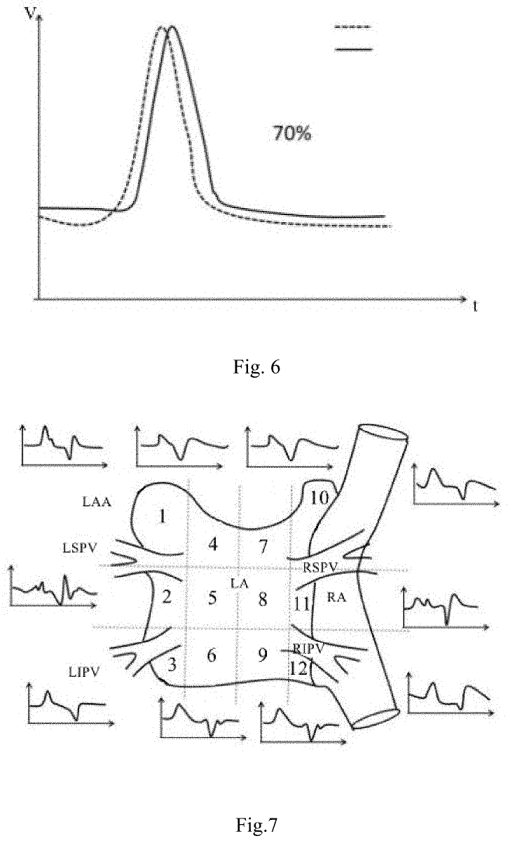

FIG. 6 schematically depicts a comparison drawn between a signal profile and an ablation pattern in accordance with an embodiment of the present invention. Referring to FIGS. 1 and 6, the waveform comparison unit 122 may calculate a degree of waveform similarity between the signal profile L and the ablation pattern M associated with the ablation lesion 20. For example, the degree of waveform similarity between the signal profile L and the ablation pattern M associated with the ablation lesion 20 may be determined as a percentage such as 70%, or 100% when they coincides with each other. When the degree of waveform similarity is greater than a predetermined threshold (e.g., 80%, 90% or higher), the waveform comparison result output to the determining unit 123 may indicate similarity between the two. Otherwise, it will indicate dissimilarity therebetween. In order to obtain the degree of waveform similarity with higher accuracy, the signal profile L may be compared with each of available ablation patterns M and a statistical analysis may be then performed on the comparisons.

Those skilled in the art will appreciate that multiple intracardiac sites may need to be ablated in a single cardiac radiofrequency ablation procedure. With a PVI procedure as an example, it may be required to form a continuous ablation lesion circumferentially around each of one or more pulmonary veins. Preferably, according to an embodiment of the present invention, the storage module 160 in the ablation lesion assessment system 100 stores ablation patterns M for ablation lesions for various intracardiac sites. Moreover, the ablation lesion assessment system 100 may further include the pattern selection module 170 (see FIG. 4) for acquiring information about the intracardiac site where an ablation lesion 20 is being formed and selecting corresponding ablation pattern(s) M.

FIG. 7 schematically illustrates ablation lesions for different intracardiac sites and corresponding ablation patterns in accordance with embodiments of the present invention, in which, LAA: the left atrial appendage; LSPV: the left superior pulmonary vein; LIPV: the left inferior pulmonary vein; RSPV: the right superior pulmonary vein; RA: the right atrium; and RIPV: the right inferior pulmonary vein. Further, the pulmonary veins and the surrounding heart tissues may be divided into 12 sub-regions, in which the pulmonary veins are located in the sub-regions 5 and 8, while the other sub-regions (i.e., 1-4, 6, 7 and 9-12) form an annulus surrounding the pulmonary veins. In PVI procedures, ablation lesions are often formed in the surrounding sub-regions.

In preferred embodiments, during computational operations of the waveform comparison unit 122 for an ablation lesion 20, the pattern selection module 170 may first determine the intracardiac site where the ablation lesion 20 is being formed and then select ablation pattern(s) M associated with the intracardiac site. In this way, the waveform comparison unit 122 can compare a signal profile L from the electrode brought into contact with the ablation lesion 20 with the ablation pattern(s) M associated with the intracardiac site where the ablation lesion 20 is being formed and obtain a waveform comparison result. The pattern selection module 170 may be connected to a three-dimensional mapping system, which can automatically partition the atria and ventricles into sub-regions and automatically identify any of the sub-regions by a mapping catheter brought into contact therewith. With the aid of the three-dimensional mapping system, the pattern selection module 170 can acquire information about the target intracardiac site and select associated ablation pattern(s) M to which the signal profile L is compared by the waveform comparison unit 122. The functions of the three-dimensional mapping system can be performed by any suitable method known in the art, and a further detailed description thereof is omitted herein.

In summary, in accordance with embodiments of the present invention, the ablation lesion assessment system 100 may derive a proportion of positive portions of a signal profile L in the whole signal profile L from an analysis on the signal profile L by the baseline calculation unit 121, and transmit the proportion to the determining unit 123. Moreover, the ablation lesion assessment system 100 may obtain a waveform comparison result from a comparison drawn between the signal profile L and ablation pattern(s) M associated with the ablation lesion 20 by the waveform comparison unit 122, and transmit the waveform comparison result to the determining unit 123.

The ablation lesion assessment system 100 may choose either the baseline calculation unit 121 or the waveform comparison unit 122 to analyze the signal profile L, and provide the result to the determining unit 123. Alternatively, the baseline calculation unit 121 and the waveform comparison unit 122 may be chosen to analyze the signal profile L successively or simultaneously, and the respective results may be then sent to the determining unit 123.

Specifically, in some embodiments, upon receiving the proportion of positive portions in the whole signal profile L from the baseline calculation unit 121, the determining unit 123 may determine whether the proportion is equal to 100%, which indicates that the signal profile L is wholly above the baseline with all negative portions of the signal profile L having turned positive. If so, it may be determined that complete ablation has been achieved, and ablation information indicative of the success may be output. Otherwise, ablation information indicating that complete ablation has not been achieved may be output.

In other embodiments, when receiving the waveform comparison result between the signal profile L and the ablation pattern(s) M from the waveform comparison unit 122, the determining unit 123 may determine whether the waveform comparison result indicates similarity that means achieved complete ablation or dissimilarity that means unachieved complete ablation.

In some other embodiments, the determining unit 123 may make a determination based on both the proportion from the baseline calculation unit 121 and the waveform comparison result from the waveform comparison unit 122. In this case, ablation information indicating that complete ablation has been achieved may be output only when both of the proportion and waveform comparison result are satisfactory. Otherwise, ablation information indicating that complete ablation has not been achieved yet may be output.

The ablation information output from the determining unit 123 may be in the form of text. Moreover, all of the proportion, the degree of waveform similarity, the waveform comparison result and the ablation information may be displayed on the display module 150. For example, the ablation information output before the achievement of complete ablation may be displayed as “In Ablation”, “Ablation in Progress” or “Ablation Not Completed”, and that output after complete ablation has been achieved may be displayed as “Ablation Completed” or “Ablation Ceased”. Alternatively, the ablation information output from the determining unit 123 may be in the form of a voice message saying, for example, “Ablation completed” or “Ablation Ceased” when the determining unit 123 determines that complete ablation has been achieved. In this case, the ablation lesion assessment system may also include a voice module, which is coupled to the determining unit and configured to play the voice message.

When determining that the proportion is equal to 100% and/or that the degree of waveform similarity is greater than a threshold, the determining unit 123 may output the ablation information indicating that complete ablation has been achieved to the display module 150 or voice module not immediately but after the elapse of (waiting for) a period of time which may be preconfigured at the instruction reception module 130 either manually or automatically. This can achieve the advantage of an enhanced probability of complete cell necrosis and a minimized chance of reversible damage. Here, “reversible damage” refers to damage that is created during a radiofrequency ablation procedure but is repaired later due to self-healing capability of the tissue. Reversible damage may lead to postoperative electrical reconnection and is therefore a condition that should be avoided as much as possible. The predetermined period of time may be, for example, 1-10 seconds or longer, preferably 2 seconds. It should be noted that if the predetermined period of time is too long, in addition to ablation lesion 20, it may cause excessive ablation and bring damage to other organs.

The ablation lesion assessment system 100 offers at least the following advantages. First, the baseline calculation unit 121 is able to identify positive portions of a signal profile L and calculate a proportion of them in the whole signal profile L, which can be displayed to provide the surgeon with a convenient real-time indication of how the ablation is carrying on. In other words, when each negative wave of the signal profile L has turned positive, the surgeon can know that the ablation lesion is transmural. This can ensure both surgery validity and significantly enhanced safety of the procedure. Second, the waveform comparison unit 122 is able to compare the signal profile L with the ablation pattern(s) M in real time and thereby obtain a waveform comparison result, which also provides the surgeon a convenient real-time indication of how the ablation is carrying on. Third, it is preferred to compare multiple ablation patterns M with the real-time signal profile L and obtain the waveform comparison result as the average or median of the resulting Pearson correlation coefficients. Alternatively, the waveform comparison result may be obtained as a degree of waveform similarity between the signal profile L and multiple ablation patterns M that form a statistical sample group. This can minimize or avoid any error that may rise from the use of a single ablation pattern. Fourth, a waveform comparison result with higher accuracy can be obtained by using a neural network algorithm that has been trained with clinical unipolar electrocardiograms as the ablation patterns M. Fifth, in order to provide the surgeon with a more accurate indication, different ablation pattern(s) M may be chosen for intracardiac electrode signals from different intracardiac sites to obtain the waveform comparison result.

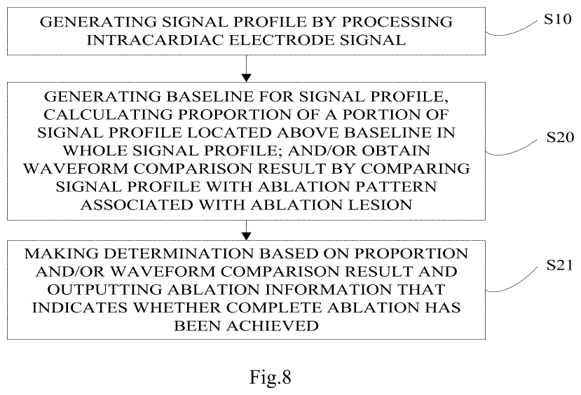

In accordance with embodiments of the present invention, there is also provide an ablation lesion assessment method for obtaining ablation information by processing an intracardiac electrode signal from an electrode brought into contact with the ablation lesion. FIG. 8 is a flowchart of this method, which includes the steps of:

S10: generating a signal profile by processing the intracardiac electrode signal; S20: generating a baseline for the signal profile and calculating a proportion of portions of the signal profile located above the baseline (i.e., positive portions) in the whole signal profile; and/or obtaining a waveform comparison result by comparing the signal profile with ablation pattern(s); and

S21: making a determination based on the proportion and/or the waveform comparison result and outputting ablation information that indicates whether complete ablation has been achieved.

This method can be implemented by the above-described system 100. For example, step S10 can be carried out by the signal processing module 110 in the system, while steps S20 and S21 can be performed by the signal analysis module 120.

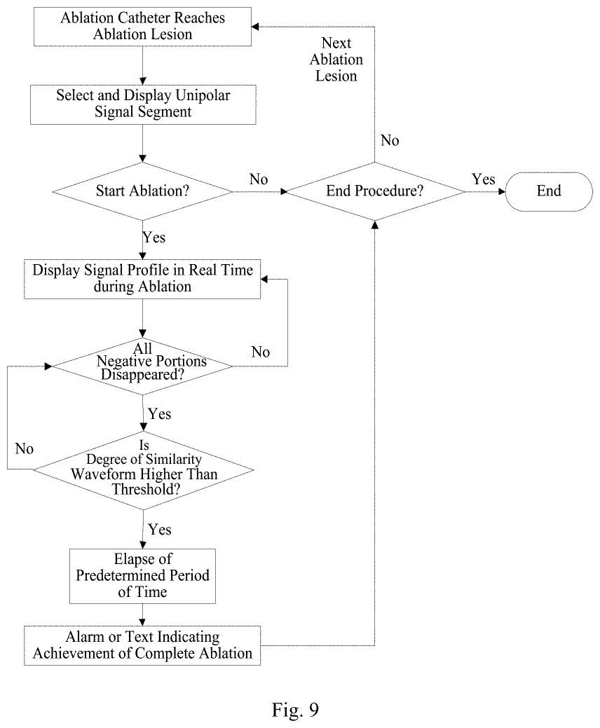

FIG. 9 schematically illustrates the use of the ablation lesion assessment method in an ablation process in accordance with an embodiment of the present invention. Referring to FIG. 9, for example, the ablation process may be a cardiac radiofrequency ablation procedure in which the electrode brought into contact with the surface of the ablation lesion is disposed at a distal end of an ablation catheter and the intracardiac electrode signal from the electrode experiences processes such as filtering and selection of a unipolar signal segment, resulting in the formation of the signal profile. At this point, it is determined whether to start ablation. If “Yes”, the ablation catheter is activated to ablate the tissue, concurrently with the signal profile being refreshed at a predetermined rate and displayed. Specifically, a proportion of portions of the signal profile located above the baseline in the whole signal profile may be calculated, and when it reaches or exceeds 100% (i.e., all negative portions have disappeared or turned positive), a waveform comparison result may be obtained by comparing the signal profile with ablation pattern(s) associated with the ablation lesion. For example, a degree of waveform similarity may be calculated between the signal profile and the ablation pattern(s), and when this value is greater than a predetermined threshold, information indicating the achievement of complete ablation in the form of an alarm or text may be output after a predetermined period of time elapses. For example, the information indicating that complete ablation has been achieved may be displayed on a display device.

In this method, ablation information indicating whether complete ablation has been achieved can be obtained from analysis and processing of an intracardiac electrode signal from an electrode brought into contact with an ablation lesion and provided to the surgeon. For example, when all negative portions have turned positive, an indication of complete transmurality of the tissue being ablated may be provided. In this way, the reliance on the surgeon's experience can be minimized or eliminated, helping the surgeon know the progress of an intracardiac radiofrequency ablation procedure. Since this method has assessment features corresponding to those of the above-described ablation lesion assessment system, it has been described in a simplified way, and reference can be made to the above description of the ablation lesion assessment system for details in such assessment features. This method allows assessment of the information about the ablation lesion with which the electrode is brought into contact before, during and after ablation, thus resulting in a significantly increased success rate and safety of cardiac radiofrequency ablation.

Processing and execution in the foregoing embodiments are generally accomplished by the combination of a software program and hardware. However, they can also be implemented in part or wholly by electronic hardware. Whether by way of software or hardware, some portions are implementable to persons familiar with the fields of electronics and software and are thus not described in detail herein. The software program can be stored on a computer readable medium such as a CD-ROM or a memory in a computer system. When loaded into a computer, instructions of the software can be executed by a central processing unit (CPU).

The description presented above is merely that of some preferred embodiments of the present invention and does not limit the scope thereof in any sense. Any person skilled in the art can make possible changes and modifications to the subject matter of the present invention based on the above teachings without departing from the spirit and scope of the present invention. Accordingly, any and all simple changes, equivalent alternatives and modifications made to the foregoing embodiments based on the essence of the present invention without departing the scope of the invention fall within the scope thereof.

1. An ablation lesion assessment system for obtaining an ablation information by processing and analyzing an intracardiac electrode signal from an ablation lesion, the ablation lesion assessment system comprising:
a signal processing module configured to process the intracardiac electrode signal and to generate a signal profile; and
a signal analysis module comprising a determining unit, wherein the signal analysis module further comprises one or both of a baseline calculation unit and a waveform comparison unit, wherein:
the baseline calculation unit is configured to generate a baseline for the signal profile, calculate a proportion of a portion of the signal profile located above the baseline in the whole signal profile and feed back the proportion to the determining unit,
the waveform comparison unit is configured to obtain a waveform comparison result by comparing the signal profile with an ablation pattern associated with the ablation lesion, and feed back the waveform comparison result to the determining unit,
the determining unit is configured to make a determination based on the proportion and/or the waveform comparison result and output the ablation information that indicates whether a complete ablation has been achieved.

2. The ablation lesion assessment system of claim 1, wherein the intracardiac electrode signal varies over time, and wherein the signal processing module is configured to obtain and process the intracardiac electrode signal in real time at a predetermined refresh rate and thereby generate a corresponding real-time signal profile.

3. The ablation lesion assessment system of claim 2, wherein the predetermined refresh rate is 1-2000 Hz.

4. The ablation lesion assessment system of claim 1, wherein when the ablation information indicates that the complete ablation has been achieved, the determining unit is configured to output the ablation information with a delay of a predetermined period of time after the determination is made.

5. The ablation lesion assessment system of claim 4, wherein the predetermined period of time is 1-10 seconds.

6. The ablation lesion assessment system of claim 1, wherein when the proportion is equal to 100%, the ablation information indicates that the complete ablation has been achieved, otherwise indicates that the complete ablation has not been achieved; or wherein when the waveform comparison result indicates a similarity, the ablation information indicates that the complete ablation has been achieved, otherwise indicates that the complete ablation has not been achieved.

7. The ablation lesion assessment system of claim 1, wherein when the proportion is equal to 100%, the waveform comparison unit feeds back the waveform comparison result to the determining unit, and when the waveform comparison unit indicates a similarity, the ablation information indicates that the complete ablation has been achieved and otherwise indicates that the complete ablation has not been achieved.

8. The ablation lesion assessment system of claim 7, wherein the waveform comparison unit is configured to calculate a degree of waveform similarity between the signal profile and the ablation pattern associated with the ablation lesion, and when the degree of waveform similarity is greater than a predetermined threshold, the waveform comparison result indicates the similarity and otherwise indicates a dissimilarity.

9. The ablation lesion assessment system of claim 7, wherein the ablation lesion is associated with a plurality of ablation patterns, and the waveform comparison unit is configured to calculate a Pearson correlation coefficient between the signal profile and each of the plurality of ablation patterns and obtain an overall Pearson correlation coefficient by taking an average or a median of a plurality of Pearson correlation coefficients, wherein when the overall Pearson correlation coefficient is 0.6-1, the waveform comparison result indicates the similarity, otherwise indicates a dissimilarity, and wherein each of the plurality of Pearson correlation coefficients is calculated according to
$$P ( X , Y ) = \frac{Cov ( X , Y )}{{σ}_{X} {σ}_{Y}} ,$$
where P (X, Y) represents the Pearson correlation coefficient, X and Y denote X and Y coordinates of the signal profile and the ablation pattern, Cov(X,Y) is a covariance of X and Y, and σ_{X} and σ_{Y} are standard deviations of X and Y.

10. The ablation lesion assessment system of claim 1, wherein the ablation lesion is associated with a plurality of ablation patterns, and the waveform comparison unit is configured to obtain the waveform comparison result by comparing the signal profile with the plurality of ablation patterns using a neural network algorithm.

11. The ablation lesion assessment system of claim 1, further comprising one or more of:
an instruction reception module configured to activate or deactivate the ablation lesion assessment system and to set parameters of the ablation lesion assessment system;
a signal input module configured to obtain and transmit the intracardiac electrode signal to the signal processing module;
a display module configured to display the ablation information;
a voice module configured to convey the ablation information;
a storage module configured to store the ablation pattern; and
a pattern selection module configured to obtain an intracardiac site where the ablation lesion is being formed and to select an associated ablation pattern.

12. The ablation lesion assessment system of claim 1, wherein the intracardiac electrode signal is a single-electrode signal obtained by one electrode that is attached to an ablation catheter or an intracardiac mapping catheter and is brought into contact with the ablation lesion.

13. An ablation lesion assessment method for obtaining an ablation information by analyzing an intracardiac electrode signal from an ablation lesion, comprising the steps of:
generating a signal profile by processing the intracardiac electrode signal;
generating a baseline for the signal profile, and calculating a proportion of a portion of the signal profile located above the baseline in a whole signal profile; and/or obtaining a waveform comparison result by comparing the signal profile with an ablation pattern associated with the ablation lesion; and
making a determination based on the proportion and/or the waveform comparison result and outputting the ablation information that indicates whether a complete ablation has been achieved.

14. The ablation lesion assessment method of claim 13, wherein making the determination based on the proportion and/or the waveform comparison result comprises:
when the proportion is equal to 100%, the ablation information indicates that the complete ablation has been achieved, otherwise indicates that the complete ablation has not been achieved; or when the waveform comparison result indicates a similarity, the ablation information indicates that the complete ablation has been achieved, otherwise indicates that the complete ablation has not been achieved.

15. The ablation lesion assessment method of claim 13, wherein making the determination based on the proportion and/or the waveform comparison result comprises:
when the proportion is equal to 100%, the waveform comparison result is determined, and when the waveform comparison result indicates a similarity, the ablation information indicates that the complete ablation has been achieved, otherwise indicates that the complete ablation has not been achieved.

16. The ablation lesion assessment system of claim 13, wherein when the ablation information indicates that the complete ablation has been achieved, outputting the ablation information a predetermined period of time after the determination is made.
